(19) United States
(12) Patent Application Publication
(10) Pub. No.: US 2012/0158153 A1
(43) Pub. Date: Jun. 21, 2012

(54) TRAPEZIUM PROSTHESIS

(75) Inventors: Frederick H. Hardenbrook, Egg Harbor Township, NJ (US); Seth D. Dodds, Hamden, CT (US); Mark Mellon, Spotswood, NJ (US)

(73) Assignee: REPLICATION MEDICAL INC., Cranbury, NJ (US)

(21) Appl. No.: 13/380,284

(22) PCT Filed: Jun. 23, 2010
(86) PCT No.: PCT/US10/39658

§ 371 Date: Feb. 27, 2012

Related U.S. Application Data
(60) Provisional application No. 61/219,709, filed on Jun. 23, 2009.

(51) Int. Cl. A61F 2/42

(52) U.S. Cl. 623/21.12

(57) ABSTRACT

A trapezium prosthesis is provided which includes a body having a frustocylindrical shape, the frustocylindrical shape including a frustum and an oppositely disposed planar base, the frustum being canted relative to a planar base. The prosthesis may be made of a swellable fluid absorbing polymer which imparts flexibility and/or resiliency to the prosthesis. The swellable fluid absorbing polymer may also provide the surface of the prosthesis with lubricity. In embodiments, the prosthesis has a first configuration and a second configuration, the first configuration being of reduced size relative to the second configuration. The prosthesis may also include a reinforcement member. In embodiments, the prosthesis includes a stem projecting outwardly from the frustum.

### CROSS-REFERENCE TO RELATED APPLICATION

This application claims priority of U.S. Provisional Application Ser. No. 61/219,709, filed Jun. 23, 2009 and is incorporated herein by reference in its entirety.

### BACKGROUND

1. Field of the Invention

The present disclosure is directed to prosthetic implants for replacement of a trapezium.

2. Description of Related Art

The basal joint of the thumb, the carpometacarpel (CMC) joint, is one of the joints most commonly affected by arthritis. Until recently the typical surgical treatment for this condition involved a large incision, excision of the entire trapezium and interposition of a tendon harvested from the forearm. Total trapezium replacements or trapezial implants made of materials such as silicone, titanium, zirconia, and pyrocarbon have been developed but with potential side effects, including conditions such as silicone sinovitis and subluxation of the joint. There exists a need for a dynamic, highly biocompatible trapezium replacement that can reduce or prevent the known side effects of artificial trapezium replacement therapies while preserving the natural anatomy.

### SUMMARY

A trapezium prosthesis is provided including a body having a frustocylindrical shape, the frustocylindrical shape including a linear frustum and an oppositely disposed planar base, the frustum being canted relative to a planar base. In embodiments, a swellable, resilient, flexible trapezium prosthesis is provided that is made of a swellable fluid absorbing polymeric medium, the prosthesis being dimensioned and configured to fit into the space vacated by removal of the native trapezium. In embodiments, the prosthesis made of a swellable fluid absorbing polymeric medium has a frustocylindrical shape. In embodiments, the prosthesis made of a swellable fluid absorbing polymeric medium has a frustum which is canted relative to a planar base. In embodiments, the polymeric medium is a hydrogel. In embodiments, the prosthesis contains an internal reinforcement member. In embodiments, the internal reinforcement member is dimensioned and configured to receive and support a fastener for affixing the prosthesis to surrounding hard or soft tissue. In embodiments, the internal reinforcement member is pre-attached to a fastener which extends outwardly from the prosthesis, the fastener adapted and configured for attaching to surrounding hard or soft tissue for purposes of stabilizing the position of the prosthesis in the implant cavity. In embodiments, the prosthesis includes a stem projecting outwardly from the frustum, the stem being dimensioned and configured to project into a canal in metacarpal bone to provide fixation of the prosthesis.

### BRIEF DESCRIPTION OF THE DRAWINGS

FIG. 1 is a dorsal view of a human skeletal hand.

FIG. 2 is a side view of a trapezium prosthesis according to the present disclosure.

FIG. 3 is a perspective view of an embodiment of a trapezium prosthesis according to the present disclosure.

FIG. 4 is a side view of a trapezium prosthesis according to the present disclosure.

FIG. 5 is a perspective view of an embodiment of a trapezium prosthesis according to the present disclosure.

FIG. 6 is a dorsal view of a human skeletal hand showing placement of a trapezium prosthesis according to the present disclosure.

### DETAILED DESCRIPTION OF PREFERRED EMBODIMENTS

A swellable, resilient, flexible trapezium prosthesis according to the present diclosure is utilized to replace the trapezium bone in a CMC joint, e.g., in a patient suffering from a severe case of osteoarthritis, from trauma or for any other reason requiring replacement or augmentation of the trapezium. The prosthesis may be delivered in a hydrated state allowing for immediate visualization of the joint space prior to closing of the incision. Alternatively, the prosthesis may be at least partially dehydrated, thus providing a reduced dimensional aspect which swells to final dimensions upon hydration. In this manner, a smaller incision may be utilized, thus reducing the amount of surgically induced trauma at the implantation site. The ability to swell to an expanded configuration in situ allows the prosthesis to partially or substantially fill the required anatomical space and mimic the shape and function of the original physiological architecture. For example, an expanded prosthesis is dimensioned and configured to provide proper joint spacing and excellent intermedullary bone fixation. In embodiments, the prosthesis is provided in an anatomically friendly, frusto-cylindrical shape to induce proper positioning of the thumb (first) metacarpal bone and reduce chance of joint dislocation due to prosthesis malfunction.

A hydrogel trapezium prosthesis according to the present disclosure provides an improvement over existing implants in that its high biocompatibility will decrease the occurrence of synovitis at the joint due to prosthetic particulates. The anatomic, yet simple, geometry provides excellent positioning post implantation and a potential decrease in the risk of joint subluxation. The flexibility and resilience of the prosthesis allows for the prosthesis to conform nicely to the natural joint anatomy resulting in a better patient to patient fit of the device. In embodiments, the trapezium prosthesis is frustocylindrical and includes a linear frustum which is canted relative to a planar base. The resilient surface and the canted linear frustum combine to “self-orient” and stabilize the prosthesis between the first metacarpal, scaphoid and trapezoid bones. The interaction between the canted surface and surrounding bones and ligaments causes the implant to bias inwardly and resist the joint's natural tendency to slide apart leading to a painful dislocation of the thumb. The resilient nature of the prosthesis allows it to conform to natural variations in anatomy from patient to patient and to exert force against the surrounding bones and thus, along with the canted surface, stabilizes the prosthesis in the desired anatomical space. The linear surface of the frustum receives adjacent bone which indents into the surface of the frustum. The indentation exerts a stabilizing force against the bone while cradling it with a custom fit. This provides an advantage over fixed surfaces which, even if, e.g., concave, would wear over time, thus allowing the prosthesis to slide out and become dislocated.

In embodiments, a hydrogel herein provides a lubricious surface which provides the prosthesis with a relatively frictionless surface, which reduces wear of surrounding tissue and the prosthesis itself. The lubricious surface combined with the geometry of the prosthesis provide an ideal modality to lock the prosthesis in place while allowing unimpeded sliding motion as required in so complex a joint.

In embodiments, a stemless configuration allows the surgeon to better preserve the thumb metacarpal resulting in a less invasive procedure and potentially decreasing surgery time and patient trauma. In embodiments, a swellable, resilient trapezium prosthesis according to the present disclosure may be implanted using minimally invasive surgery as a result of the ability of the prosthesis to achieve an optimum implantable substantially reduced configuration and further ability to expand anisotropically or isotropically to an expanded configuration which is adapted and configured to at least partially fill the desired implant space.

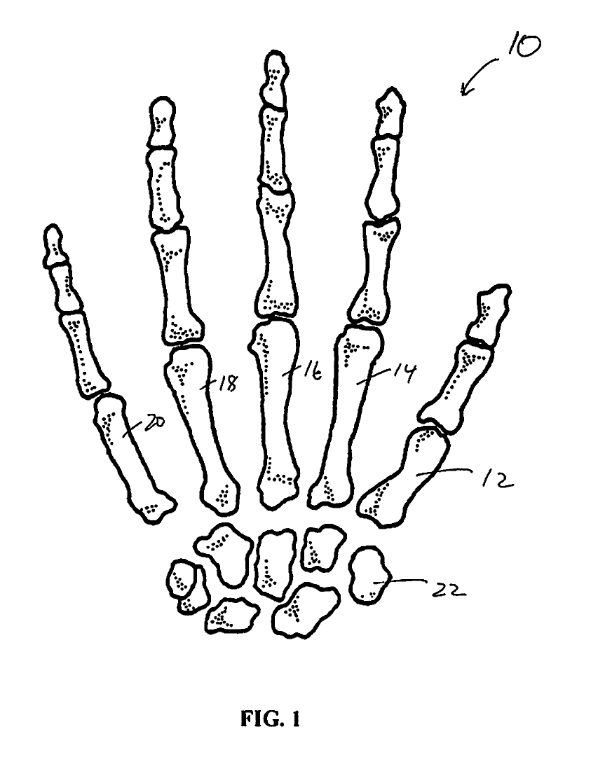

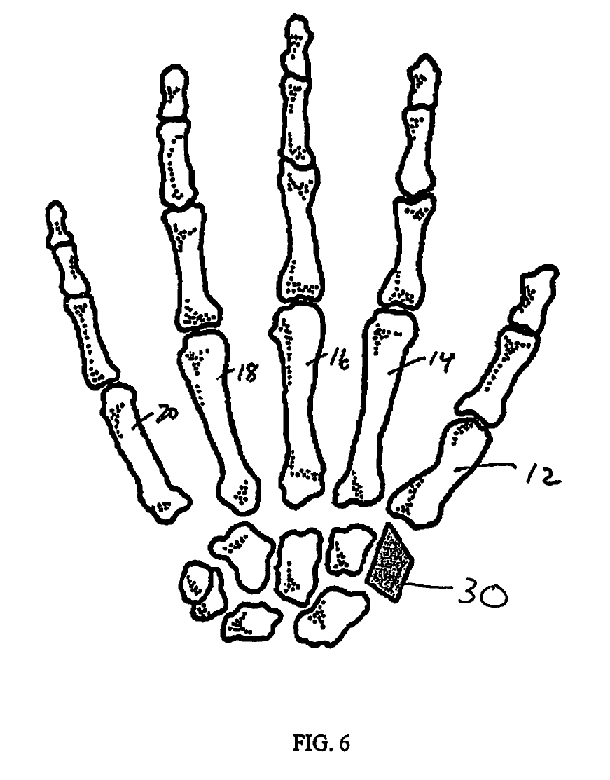

FIG. 1 depicts a dorsal view of a human skeletal left hand 10 which includes a first metatarsal 12 (thumb), a second metatarsal 14, a third metatarsal 16, a fourth metatarsal 18 and a fifth metatarsal, 20. The hand also includes a carpus including a trapezium 22. Prostheses, according to the present disclosure, are utilized to replace the native trapezium and are dimensioned and configured to occupy the space vacated by removal of the native trapezium. See for example, FIG. 6 which depicts a trapezium prosthesis 30 implanted in the skeletal hand 10.

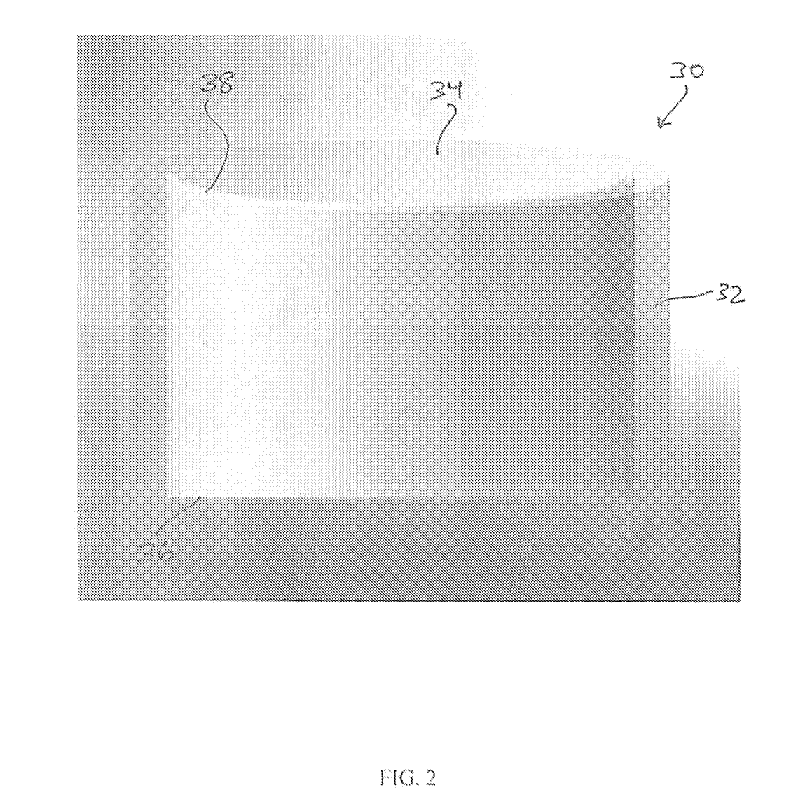

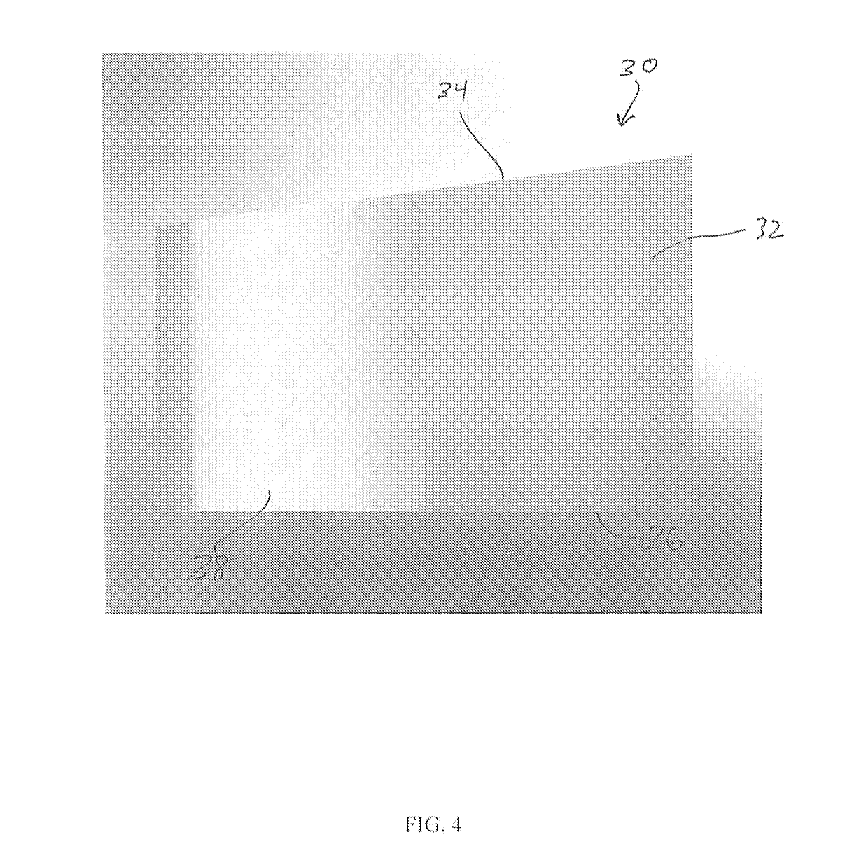

Referring to FIGS. 2, 3 and 4, in embodiments, the trapezium prosthesis 30 is frusto-cylindrical and includes an implant body 32 and a frustum 34 which is canted relative to a planar base 36. An optional interiorly disposed reinforcement member 38 is shown in a semicircular configuration near the perimeter of the prosthesis 30. In embodiments, the reinforcement member 38 may be utilized as a buttress for fixation of a fastener, e.g., for suture fixation, staple fixation, screw fixation, barb fixation and the like. The reinforcement member 38 allows for improved fastener retention by the implant for long term placement. Fastener fixation allows for preservation of the metacarpal bone of the thumb by removing the need for a metacarpal canal for stem placement.

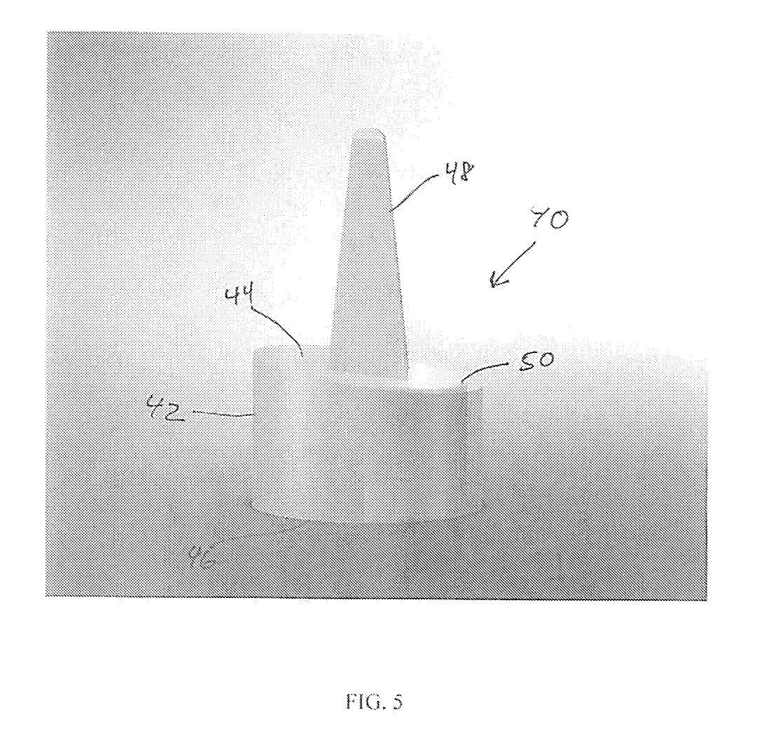

In embodiments, a trapezium prosthesis incorporates a stem as shown, e.g., in FIG. 5. A trapezium prosthesis 40 includes an implant body 42 and a frustum 44 which is canted relative to a planar base 46. The stem 48 projects outwardly from the frustum and is dimensioned and configured to be inserted into a canal formed in the first metatarsal 12 to provide fixation and allow for proper placement of the prosthesis. An optional interiorly disposed reinforcement member 50 is shown in a semicircular configuration near the perimeter of the prosthesis 40. In embodiments, the reinforcement member 50 may be utilized as a buttress for fixation of a fastener, e.g., for suture fixation, staple fixation, screw fixation, barb fixation and the like. The reinforcement member 50 allows for improved fastener retention by the implant for long term placement.

When fully hydrated, the swellable, flexible trapezium prosthesis is resilient enough to hold its shape despite the rigors and stress of normal hand movements. In embodiments, the surface of the prosthesis is lubricious which helps prevent wear on the prosthesis as well as to surrounding bones, tendons and ligaments. In embodiments, the chemical make-up of the swellable polymer can be altered to make the portions of, or the entire prosthesis, softer or stiffer, i.e., more or less elastic. It is also contemplated that non-swellable polymers may be utilized when the frustum 34 which is canted relative to a planar base 36. Examples include polyethylene, polyethylene terephthalate, polycarbonate and methacrylate polymers and copolymers. Metals such as titanium may also be used.

Fluid absorbing polymers are well-suited for manufacturing a swellable, resilient trapezium prosthesis in accordance with the present disclosure. Suitable fluid absorbing polymers include synthetic polymers such as poly(ethylene glycol), poly(ethylene oxide), partially or fully hydrolyzed poly(vinyl alcohol), poly(vinylpyrrolidone), poly(ethyloxazoline), poly(ethylene oxide)-co-poly(propylene oxide) block copolymers (poloxamers and meroxapols), poloxamines, carboxymethyl cellulose, and hydroxyalkylated celluloses such as hydroxyethyl cellulose and methylhydroxypropyl cellulose, and natural polymers such as polypeptides, polysaccharides or carbohydrates such as Ficoll™, polysucrose, hyaluronic acid, dextran, heparan sulfate, chondroitin sulfate, heparin, or alginate, and proteins such as gelatin, collagen, albumin, or ovalbumin or copolymers or blends thereof. As used herein, “celluloses” includes cellulose and derivatives of the types described above; “dextran” includes dextran and similar derivatives thereof. Examples of materials that can be used to form a hydrogel include modified alginates. Alginate is a carbohydrate polymer isolated from seaweed, which can be crosslinked to form a hydrogel by exposure to a divalent cation such as calcium. Alginate is ionically crosslinked in the presence of divalent cations, in water, at room temperature, to form a hydrogel matrix. Modified alginate derivatives may be synthesized which have an improved ability to form hydrogels.

Additionally, polysaccharides which gel by exposure to monovalent cations, including bacterial polysaccharides, such as gellan gum, and plant polysaccharides, such as carrageenans, may be crosslinked to form a hydrogel using methods analogous to those available for the crosslinking of alginates described above. Polysaccharides which gel in the presence of monovalent cations form hydrogels upon exposure, for example, to a solution comprising physiological levels of sodium. Hydrogel precursor solutions also may be osmotically adjusted with a nonion, such as mannitol, and then injected to form a gel.

Other polymeric hydrogel precursors include polyethylene oxide-polypropylene glycol block copolymers such as Pluronics™ or Tetronics™, which may be crosslinked by hydrogen bonding and/or by a temperature change. Other materials which may be utilized include proteins such as fibrin, collagen and gelatin. Polymer mixtures also may be utilized. For example, a mixture of polyethylene oxide and polyacrylic acid which gels by hydrogen bonding upon mixing may be utilized. In one embodiment, a mixture of a 5% w/w solution of polyacrylic acid with a 5% w/w polyethylene oxide (polyethylene glycol, polyoxyethylene) 100,000 can be combined to form a gel over the course of time, e.g., as quickly as within a few seconds.

Water soluble polymers with charged side groups may be crosslinked by reacting the polymer with an aqueous solution containing ions of the opposite charge, either cations if the polymer has acidic side groups or anions if the polymer has basic side groups. Examples of cations for cross-linking of the polymers with acidic side groups to form a hydrogel are monovalent cations such as sodium, divalent cations such as calcium, and multivalent cations such as copper, calcium, aluminum, magnesium, strontium, barium, and tin, and di-, tri- or tetra-functional organic cations such as alkylammonium salts. Aqueous solutions of the salts of these cations are added to the polymers to form soft, highly swollen hydrogels and membranes. The higher the concentration of cation, or the higher the valence, the greater the degree of cross-linking of the polymer. Additionally, the polymers may be crosslinked enzymatically, e.g., fibrin with thrombin. The polymers can be covalently crosslinked as well through the addition of ethylene diamine, NBS or a host of crosslinking agents routinely to react with amino, nitrile, urethane and carboxylic functional groups found on the polymer chain.

Suitable ionically crosslinkable groups include phenols, amines, imines, amides, carboxylic acids, sulfonic acids and phosphate groups. Negatively charged groups, such as carboxylate, sulfonate and phosphate ions, can be crosslinked with cations such as calcium ions. The crosslinking of alginate with calcium ions is an example of this type of ionic crosslinking. Positively charged groups, such as ammonium ions, can be crosslinked with negatively charged ions such as carboxylate, sulfonate and phosphate ions. Preferably, the negatively charged ions contain more than one carboxylate, sulfonate or phosphate group.

Anions for cross-linking of the polymers to form a hydrogel are monovalent, divalent or trivalent anions such as low molecular weight dicarboxylic acids, for example, terepthalic acid, sulfate ions and carbonate ions. Aqueous solutions of the salts of these anions are added to the polymers to form soft, highly swollen hydrogels, as described with respect to cations.

A variety of polycations can be used to complex and thereby stabilize the polymer hydrogel into a semi-permeable surface. Examples of materials that can be used include polymers having basic reactive groups such as amine or imine groups, having a preferred molecular weight between 3,000 and 100,000, such as polyethylenimine and polylysine. These are commercially available. One polycation is poly(L-lysine); examples of synthetic polyamines are: polyethyleneimine, poly(vinylamine), and poly(allyl amine). There are also natural polycations such as the polysaccharide, chitosan.

In embodiments, the trapezium prosthesis is made of a hydrogel. Prior to coagulation, the liquid form of a suitable hydrogel is used to form the expanded configuration as it would be in the hydrated state. The hydrogel is then coagulated to form the prosthesis in an expanded configuration. The prosthesis may then dehydrated to a xerogel state which reduces the volume of the prosthesis to the reduced configuration. Many hydrogel polymers behave in a similar manner, which is to say they can be deformed, frozen into a deformed shape and they can maintain that shape indefinitely or until, e.g., a temperature change causes the polymer to “relax” into the shape originally held prior to freezing. This property can be referred to as shape memory or frozen deformation by those skilled in the art.

The temperature at which frozen deformation occurs is referred to as the glass transition temperature or T_{g}. At T_{g} several polymer properties such as density, entropy and elasticity may sharply change. Many polymers can be mixed with agents that can have a drastic effect on a polymer T_{g}. Polymers which absorb fluid are of particular interest and water is the preferred T_{g} altering agent. Hydrogels which contain less than about five percent water may be considered dehydrated or xerogels. The T_{g} of a xerogel will change as it absorbs fluids containing water. Once the T_{g} becomes lower than ambient the now partially hydrated hydrogel becomes pliant and may be elastically deformed. If the polymer is held in a state of elastic deformation while the T_{g} is raised above ambient the polymer will maintain the deformed state indefinitely. This can be accomplished by either lowering the ambient temperature (freezing) or by returning the polymer to its xerogel state thus raising the T_{g}.

Using this method, hydrogel articles may be produced with vastly differing xerogel shapes compared to hydrated shapes. This is especially useful in cases such as medical implants where, in delivering a prosthesis into the human body, every care should be taken to reduce trauma to the patient. A prosthesis which is shaped, e.g., as the prostheses shown in any of FIGS. 2, 3, 4 and 5 can be re-shaped into any compressed shape including, e.g., a tapered elongate rod, folded in a “C” shape, an accordion shape, and the like in order to facilitate minimally invasive implantation. Alternatively, a portion of the prosthesis can be compressed as compared to another portion of the prosthesis. Indeed, various frozen shapes may be utilized to facilitate implantation and situation of the prosthesis. Once the prosthesis is indwelling and has absorbed water containing liquids it will substantially return to the expanded shape and maintain that shape indefinitely. It should be understood that, in embodiments, various shapes may be cast to adjust to a particular patient's anatomy. As used herein, “substantially” is intended to mean any of “approximately”, “nearly” or “precisely.”

A preferred polymer configuration includes two polymer phases of different hydrophilicity, the less hydrophilic phase having higher content of hydrophobic groups and more hydrophilic phase having higher content of hydrophilic groups. The less hydrophilic phase is preferably crystalline and more hydrophilic phase is preferably amorphous, as can be established from X-ray diffraction.

Advantageous hydrophobic groups are pendant nitrile substituents in 1,3 positions on a polymethylene backbone, such as poly(acrylonitrile) or poly(methacrylonitrile). The hydrophilic phase may preferably contain a high concentration of ionic groups. Preferred hydrophilic groups are derivatives of acrylic acid and/or methacrylic acid including salts, acrylamidine, N-substituted acrylamidine, acrylamide and N-substituted acryl amide, as well as various combinations thereof. A particularly preferred combination contains approximately two thirds acrylic acid and its salts (on molar basis), the rest being a combination of plain and N-substituted acrylamides and acrylamidines.

At least one polymeric component is preferably a multiblock copolymer with alternating sequences of hydrophilic and hydrophobic groups. Such sequences are usually capable of separating into two polymer phases and form strong physically crosslinked hydrogels. Such multiblock copolymers can be, for example, products of hydrolysis or aminolysis of polyacrylonitrile or polymethacrylonitrile and copolymers thereof. For convenience, polymers and copolymers having at least about 80 molar % of acrylonitrile and/or methacrylonitrile units in their composition may be referred to as “PAN”. Hydrolysis and aminolysis of PAN and products thereof are described, for example, in U.S. Pat. Nos. 4,107,121; 4,331,783; 4,337,327; 4,369,294; 4,370,451; 4,379,874; 4,420,589; 4,943,618, and 5,252,692, each being incorporated herein by reference in their respective entireties.

A preferred fluid absorbing polymer for the trapezium prosthesis herein is a synthetic composite of a cellular (or domain) type with continuous phase formed by a hydrophobic polymer or a hydrophilic polymer with low to medium water content forming a “closed cell” spongy structure that provides a composite with good strength and shape stability. In embodiments, the fluid absorbing polymer imparts a modulus of elasticity to the prosthesis of about 1 MPa to about 10 MPa. The terms “fluid absorbing polymer”, “swellable polymer” and “swellable fluid absorbing polymer” are used interchangeably herein. Examples of suitable polymers are polyurethanes, polyureas, PAN, and highly crystalline multiblock acrylic and methacrylic copolymers. The polymer should be sufficiently permeable to water. More preferably, the continuous phase is formed by a strong hydrophilic polymer with sufficient permeability for water but impermeable to high-molecular solutes. Examples of such polymers are highly crystalline hydrogels based on segmented polyurethanes, polyvinylalcohol or multiblock acrylonitrile copolymers with derivatives of acrylic acid. Typically, suitable polymers for the continuous phase in cellular composites have a water content in fully hydrated state between about 60% by weight and about 90% by weight, preferably between about 70% and about 85% by weight.

The second component of the fluid absorbing polymer may be a highly hydrophilic polymer of high enough molecular weight to prevent permeation of the hydrophilic polymer through the continuous phase. This component is contained inside the matrix of the continuous phase. The entrapped hydrophilic polymers (the so-called “soft block”) may be high-molecular weight water-soluble polymers, associative water-soluble polymers or highly swellable hydrogels containing, in a fully hydrated state, an amount of hydration which is preferably at least about 5% greater than the hydrophobic component. For example, the second component hydrated to at least about 65% when the first component is hydrated to about 60%. In embodiments, e.g., from the second component could be fully hydrated at from about 95% of water and up to about 99.8% of water. Such hydrogels are very weak mechanically. However, it may not matter in composites where such polymers' role is generation of osmotic pressure rather than load-bearing, with e.g., compression strength in full hydration in the range of about 0.01 MN/m² or lower.

A system with closed cells (or domains) containing highly swellable or water-soluble polymers can form composites with very high swelling pressure as needed for the prosthesis anchoring function. Examples of suitable hydrophilic polymers are high-molecular weight polyacrylamide, polyacrylic acid, polyvinylpyrrolidone, polyethyleneoxide, copolymers of ethyleneoxide and propyleneoxide or hyaluronic acid; covalently crosslinked hydrogels such as hydrophilic esters or amides of polyacrylic or polymethacrylic acids; and physically crosslinked hydrogels, such as hydrolyzates or aminolysates of PAN.

Particularly suitable are associative water-soluble polymers capable of forming very highly viscous solutions or even soft physical gels. Preferred are associative polymers containing negatively charged groups, such as carboxylates, sulpho-groups, phosphate groups or sulfate groups. Particularly preferred are associative polymers formed by hydrolysis and/or aminolysis of PAN to high but finite conversions that leave a certain number of nitrile groups (typically, between about 5 and 25 molar %) unreacted.

Preferred fluid absorbing polymer composites have both a continuous phase and a dispersed phase formed by different products of hydrolysis or aminolysis of PAN. In this case, both components are compatible and their hydrophobic blocks can participate in the same crystalline domains. This improves anchorage of the more hydrophilic component and prevents its extraction or disassociation. The size of more hydrophilic domains may vary widely, from nanometers to millimeters, preferably from tens of nanometers to microns.

The ratio between the continuous discrete phase (i.e., between more hydrophobic and more hydrophilic components may vary from about 1:2 to about 1:100 on a dry weight basis, and a preferred ratio ranges from about 1:5 to about 1:20. Examples of compositions and implants are described in U.S. Pat. Nos. 6,264,695 and 6,726,721, both of which are incorporated herein by reference in their entireties. A preferred method of making the fluid absorbing polymer composite is described in U.S. Pat. No. 6,232,406, herein incorporated by reference in its entirety.

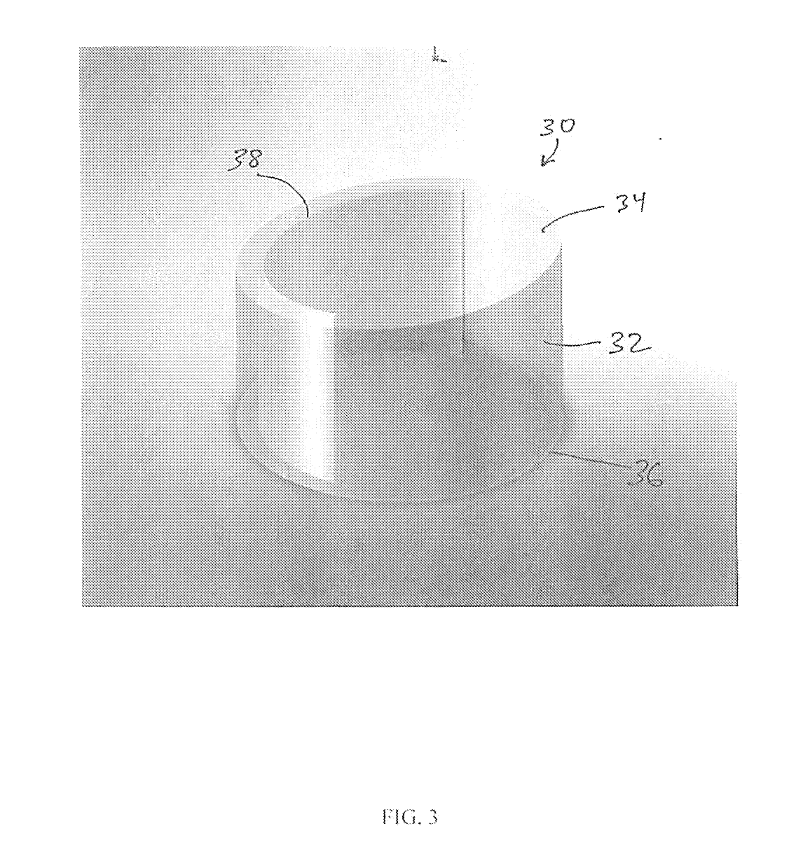

Examples of particularly suitable hydrogel forming copolymers are prepared by a partial alkaline hydrolysis of polyacrylonitrile (“HPAN”) in the presence of sodium thiocyanate (NaSCN). The resulting hydrolysis product is a multi-block acrylic copolymer, containing alternating hydrophilic and hydrophobic blocks. Hydrophilic blocks contain acrylic acid, acrylamidine, and acrylamide. In embodiments, for example, a PAN hydrolysate polymer (referred to herein HPAN I) (46±1% conversion of hydrolysis) having the following composition: acrylonitrile units ˜53-55%, acrylic acid units ˜22-24%, acrylamide units ˜17-19%, acrylamidine units ˜4-6%, as determined by ¹³C NMR, is dissolved in a suitable solvent such as a ˜55% solution of sodium thiocyanate in water to form a viscous solution. The viscous solution is poured into a porous mold having, e.g., a cavity defining the dimensions of the joint prosthesis. See, e.g., FIGS. 2 and 3. The solution can then be solvent cast, e.g., by solvent exchange (e.g., water for NaSCN). The pores should be sufficiently small as to not permit the polymer to diffuse or leak out of the mold. In another form, the hydrogel used to make the prosthesis is obtained by reacting an aquagel of PAN, formed by dissolving the polymer in an aqueous solvating solution such as high concentration of sodium thiocyanate. The resulted solution of PAN is thereupon coagulated through addition of a suitable aqueous solvent or water miscible solvent. The coagulum is further reacted in a hydrolyzing basic or acidic medium. The PAN aquagel can then be processed as a thermoplastic and molded to obtain the desired shape. These methods are described in U.S. Pat. No. 4,943,618.

A more rigid fluid absorbing polymer may be another PAN hydrosylate polymer, referred to herein as HPAN II (28±1% conversion of hydrolysis), having the following composition: acrylonitrile units ˜71-73%, acrylic acid units ˜13-15%, acrylamide units ˜10-12%, acrylamidine units ˜2-4%, as determined by ¹³C NMR, disolved in ˜55% NaSCN which can be solvent cast, washed, dried and cut to a suitable shape.

The trapezium prosthesis optionally includes an interiorly embedded reinforcement member. The reinforcement member occupies at least a portion of the interior of the prosthesis. The reinforcement member can have a planar configuration as shown, e.g., in FIGS. 2, 3, 4 and 5 or it can be one or more rods or beams of relatively rigid material which can extend for a portion of or the entire length of the prosthesis or it can consist of two or more members. A reinforcement member may be situated, e.g., in the stem. In embodiments, the reinforcement member may be made of a series of individual fibers or ribbons which are arranged in parallel or non-parallel fashion and extend throughout the prosthesis. In embodiments the reinforcement member can be a fabric or mesh. Woven or non-woven configurations are suitable. A reinforcement member may be made of a polymeric material which is natural, e.g., cotton, or synthetic, e.g., polyester, polyamide, or other materials such as metal fiber, fiber glass, and carbon fiber. Methods of making shaped objects from these materials and others are well-known to those skilled in the art. Foils or ribbons herein may also be made of metal or polymeric material and are well-known. Thus, the reinforcement member may be constructed from relatively durable materials including, but not limited to, metal foil, plastic foil, metal fibers, polymeric fibers of materials such as polycarbonate, polyethylene, polypropylene, polystyrene, polyethylene terephthalate, polyamide, polyurethane, polyurea, polysulfone, polyvinyl chloride, acrylic and methacrylic polymers, expanded polytetrafluoroethylene (Goretex®), ethylene tetrafluoroethylene, graphite, etc. These materials can be used either alone, or in a composite form in combination with elastomers or hydrogels. Alternatively, the reinforcement member may be exteriorly disposed, e.g., a jacket which surrounds all or part of the trapezium prosthesis. The reinforcement member is designed to provide support for the prosthesis and to buttress any fastener which may be used to help maintain the prosthesis in place in the space vacated by the native trapezium. Suitable fasteners include, but are not limited to sutures, screws, staples, barbs and the like. The reinforcement member may be pre-loaded with one or more fasteners or not, e.g., in the situation where a fastener is applied by the surgeon during implantation.

A trapezium prosthesis herein may be manufactured by providing a mold corresponding to the shape of the trapezium prosthesis. A fluid absorbing liquid polymer is added to the mold and is cured or fixed, e.g., by solvent casting, ionic gelation, photo-polymerization and the like, when it solidifies or coagulates. In the case of solvent casting, the mold may be made of material which is impermeable to the fluid absorbing polymer but permeable to water. The mold is placed in a water bath to extract the solvent (e.g., sodium thiocyanate) which causes the polymer to coagulate. The mold may then be opened and any remaining solvent in the prosthesis is extracted.

It is contemplated that regions of more or less modulus of elasticity and durability may be incorporated into the trapezium prosthesis. For example, it may be desirable to fashion the optional stem from a relatively more rigid fluid absorbing polymer, e.g., the portions which contact bone. If a softer or more elastic zone of fluid absorbing polymer is desired in the central portion of the prosthesis, a hydrogel such as HPAN I can be added to the portion of the mold defining the central region. It should be understood that any number of zones of varying or the same elasticity may created in this fashion. In addition, different fluid absorbing polymers can be used to create zones with different properties. If desired, an adhesive can be added between adjacent zones to insure bonding or, e.g., in the case of the HPAN polymers, the layers can be made to naturally adhere to one another. Some co-mingling of liquid fluid absorbing polymers at zone interfaces can provide for an advantageous smooth transition between layers and reduce or eliminate the need for an adhesive between layers.

A trapezium prosthesis herein may be manufactured by providing a reinforcement member of desired configuration and placing it in a mold, e.g., as shown in FIGS. 2, 3, 4 and 5 or in an area corresponding to the stem. A fluid absorbing liquid polymer is added to the mold and surrounds the reinforcement member. In one embodiment, a gap, e.g., about 1-3 mm or more, is left between one or more sides of the reinforcement member and the walls of the mold. Fluid absorbing liquid polymer is allowed to fill the gap between the mold and the reinforcement member. When the fluid absorbing polymer is cured or fixed, e.g., by solvent casting, ionic gelation, photo-polymerization and the like, it solidifies and encapsulates the reinforcement member. In the case of solvent casting, the mold may be made of material which is impermeable to the fluid absorbing polymer but permeable to water. The mold is placed in a water bath to extract the solvent (e.g., sodium thiocyanate) which causes the polymer to coagulate. The mold may then be opened and any remaining solvent in the prosthesis is extracted. If it is desired to leave one or more sides of the prosthesis open to the reinforcement member, then the desired side(s) of the reinforcement member is placed up against the wall of the mold to prevent formation of a gap for the liquid fluid absorbing polymer to fill.

In one embodiment, the fluid absorbing polymer is made to achieve a strong physical bond to the reinforcement member by incorporating an initial treatment of the reinforcement member with a relatively hydrophobic fluid absorbing polymer to create an encapsulating layer of the relatively hydrophobic fluid absorbing polymer. For example, a hydrogel such as HPAN II is applied to the reinforcement member as a 10% solution by weight in a solvent (sodium thiocyanate 55% by weight in water) and then coagulated onto the reinforcement member by solvent exchange with an aqueous solution such as water. As the polymer coagulates, it shrinks volumetrically around the reinforcement member, causing a tight physical bond to the reinforcement member. If desired, the treated reinforcement member is placed in a mold and a relatively more hydrophilic fluid absorbing polymer in the liquid state is added to create a cohesive continuous polymer matrix which surrounds the reinforcement member. For example, a 10% by weight HPAN I in a 55% by weight sodium thiocyanate solution, is added to the mold. The solvent from the HPAN I solution causes the outermost surface of the coagulated HPAN II layer surrounding the braided fibers to dissolve and allow commingling of the HPAN I and HPAN II hydrogel polymers at the surface interface which forms a strong adhesive bond when the HPAN I and commingled hydrogels are coagulated by solvent exchange. It should be understood that the reinforcement member is optional and that a mold may be filled without such a reinforcement member.

Upon completion of the solvent exchange extraction process the trapezium prosthesis may be hydrated to its fullest extent (˜90% equilibrium water content (EWC)). In this fully hydrated state the prosthesis is readily deformed under modest loads and the hydrogel, e.g., HPAN I OR HPAN II, glass transition temperature (T_{g}) is well below room temperature. This is the “relaxed” state of the prosthesis, the state to which it will return after loading below the critical level. The critical level is the point at which permanent deformation occurs and is further discussed below. In order to provide a reduced configuration (also referred to herein as the first configuration), the prosthesis may be allowed to dehydrate and enter the xerogel state. A considerable amount of the prosthesis' volume is lost when in the xerogel state as compared to the hydrated state. Advantageously, the fully hydrated trapezium prosthesis may be deformed into a desirable insertion shape and the temperature of the prosthesis is lowered below its T_{g} (near freezing point of water). Such a prosthesis is in a state of “frozen deformation” and it would retain that deformed shape indefinitely. Once the prosthesis is warmed above its T_{g}, however, the prosthesis would recover to its original memorized configuration.

The T_{g} of the hydrogel increases with decreasing water content. This characteristic is exploited by simultaneously raising the T_{g} while deforming the prosthesis into a desired shape. In other words, as the prosthesis dehydrates it is freezing the position of the polymer chains. To regain the original shape of the prostheis, the T_{g} may be lowered by hydration.

In order to obtain a rod-shape having a cross-sectional ellipsoid shape for implantation, e.g., suppository, bullet, tapered cylinder, etc., from, e.g., the configurations shown in FIGS. 2,3, 4 and 5, reduction in volume deformation is advantageously maintained radially, substantially parallel with the longitudinal axis. This is accomplished by placing the prosthesis within a radially collapsible member for exerting circumferential compression on an object, e.g., a trapezium prosthesis, contained within the member. Suitable radially collapsible members include, e.g., a flexible sleeve such as a braided sock or tube, a flexible coil, iris diaphragm, collapsible loop, etc. In a preferred embodiment, the radially collapsible member is porous or semipermeable so that water, either as liquid or as vapor, passes through the member. The collapsible member may be made of an elastic material such as rubber or neoprene fabric which has been made porous by any technique known to those skilled in the art, or a woven or non-woven mesh or braid. The collapsible member may also be made of a flexible metal having sufficient porosity to allow water to exit from the prosthesis. The collapsible member does, however, need to be stiff enough to be able to exert sufficient compressive force when tension is applied to compress the prosthesis, i.e., it should not be so elastic that it deforms without being able to exert sufficient compressive force.

In operation, the radially collapsible member exerts substantially equilateral circumferential compression on the trapezium prosthesis by substantially uniformly decreasing in diameter while contacting the prosthesis. The preferred porous nature of the collapsible member allows water from the prosthesis to escape into the surrounding environment so that the prosthesis can become dehydrated. In one embodiment, the sleeve radially collapsible member is stretched in length which causes the inner diameter to decrease, thus compressing the prosthesis, including, e.g., a reinforcement member, into a desired implantation configuration. A more complete description of a suitable radial compression process is described in U.S. application Ser. No. 11/303,767, herein incorporated by reference in its entirety.

The collapsible member is loaded in tension via any tensioning device known to one skilled in the art, e.g., a pneumatic cylinder, a hydraulic cylinder, springs, weights, pulleys, etc. The tension on the collapsible member can be precisely controlled by regulating the pressure within the tensioning device, translating into constant, controlled radial load on the trapezium prosthesis. In the case of a sleeve collapsible member, once the prosthesis is loaded into the collapsible member and the collapsible member is tensioned, three things occur: the prosthesis dehydrates, the prosthesis deforms, and the collapsible member extends. By varying the tension on the collapsible member, the length of the prosthesis can optionally be extended, thereby decreasing the minor axis and height. This can also be controlled, to some extent, by the speed of dehydration (temperature, pressure and humidity), with longer dehydration time producing longer prosthesis length and vise versa. In certain embodiments, one portion of the collapsible member is made to collapse further than other portions to define a prosthesis having one end which is relatively more compressed than the other end.

Two concerns with respect to drying time and collapsible member tension should be considered. The first is creep, which may set in if the dehydration time is extended unreasonably long (over several days). The second is permanent deformation which may occur if excessive stress is applied to the prosthesis. Both of these concerns only occur at critical point extremes which are to be avoided. Permanent deformation may occur in the hydrogel prosthesis if the soft-block domains of the polymer are displaced to a point where they cannot reorient themselves into the original lattice configuration, i.e., the memorized shape. This can happen, e.g., by either deforming the original shape so severely that many of the bonds which hold the soft-blocks in place are severed, or by heating the prosthesis sufficiently above the T_{g} to cause the soft-block domains to permanently or irrevocably assume a new configuration outside of the originally contemplated structure, which causes an undesirable change in shape. Thus, the melting point of the soft block should not be exceeded. The melting point of the soft block may vary based on the amount of water content. Such melting points may be determined by conventional techniques known to those skilled in the art. For example, at 18% hydration of HPAN I, permanent deformation is manifest at temperatures over 105° C.

In embodiments, the majority of the dehydration process can occur at room temperature over an extended period of time (e.g., 18 to 36 hours). The prosthesis can be monitored to determine the extent of dehydration and the time period adjusted accordingly. Relative humidity, air circulation, air pressure and room temperature should be controlled during this period. Especially preferred conditions are about 21° C. at 50% relative humidity under moderate airflow. Once the prosthesis has reached <˜30% water content it may be forced dry at elevated temperature, e.g., from about 25° C. to about 105° C. for typically less than about 24 hours to rapidly remove remaining water. As above, the state of dehydration may be monitored to determine if greater or lesser amounts of time are needed. When the prosthesis is substantially completely dehydrated, the prosthesis is fairly rigid in its state of frozen deformation. Alternatively, a slight degree of hydration provides some flexibility to the prosthesis. The less dehydrated, the more flexible. It is contemplated herein that “substantially dehydrated” preferably encompasses from about 12% or less, to about 30% water by weight of the prosthesis.

Upon completion of forced dehydration, the trapezium prosthesis is extremely stable in terms of shelf life, providing that it is kept dry. Even brief exposure to humidity during the sterilization process should not have significant effects. Temperatures above about 80° C. should be avoided for extended periods as this may bring the prosthesis above its T_{g} if it has absorbed some small amount of water vapor.

Surface irregularities may be present on a dehydrated compressed trapezium prosthesis which was compressed as described above by a radially collapsible member by virtue, e.g., of some extrusion of the hydrogel through pores or through interstitial spaces of the member. For example, a woven or non-woven collapsible sleeve may have interstitial spaces that allow hydrogel to extrude therein under compressive force. In addition, after radial compression, as described above, the dimensions of the prosthesis may be different than the ultimate dimensions desired by the practitioner. Both of these instances can be remedied by post-compression thermoforming of the prosthesis. In this aspect, a dehydrated, compressed prosthesis is placed within a mold which may be advantageously pre-heated to about 70-150° C., but more preferably, closer to the melting point of the polymer, e.g., about 105° C. Care must be taken to avoid subjecting the prosthesis to excess heat which causes the hydrogel to exceed its critical point, and thus causing permanent deformation of the prosthesis. If the temperature is high, the prosthesis must be quickly removed from the mold to avoid permanent deformation. The mold is machined to the exact desired final dimensions of the xerogel prosthesis and essentially irons out surface roughness to a substantially smooth surface, which is less abrasive to surrounding tissue when implanted. If desired, and if the xerogel prosthesis is compressed by a radially compressive member or by gas compression, but has not achieved, e.g., an ideal enough straight rod-like configuration, or if the ends are not sufficiently blunted or otherwise tapered, post-compression thermoforming may be utilized to fine tune the shape as well as remove any surface irregularities which may be present. Post-compression thermoforming may also be utilized to bend a prosthesis to a desired configuration, e.g., to a boomerang shape.

A trapezium prosthesis according to the disclosure herein may contain a medicinal agent. “Medicinal agent” is used in its broadest sense and it includes any substance or mixture of substances which may have any clinical use. It is to be understood that medicinal agent encompasses any drug, including hormones, antibodies, therapeutic peptides, etc., or a diagnostic agent such as a releasable dye which has no biological activity per se. Thus, in its broadest aspect, a method of delivery herein may be defined as the release of any substance for clinical use, which may or may not exhibit biological activity.

Examples of medicinal agents that can be used include anticancer agents, analgesics, anesthetics, anti-inflammatory agents, growth factors such as BMPs, antimicrobials, and radiopaque materials. Such medicinal agents are well-known to those skilled in the art. The medicinal agents may be in the form of dry substance in aqueous solution, in alcoholic solution or particles, microcrystals, microspheres or liposomes. An extensive recitation of various medicinal agents is disclosed in Goodman and Gilman, The Pharmacological Basis of Therapeutics, 10th ed. 2001, or Remington, The Science and Practice of Pharmacy, 21 ed. (2005). As used herein, the term “antimicrobial” is meant to encompass any pharmaceutically acceptable agent which is substantially toxic to a pathogen. Accordingly, “antimicrobial” includes antiseptics, antibacterials, antibiotics, antivirals, antifungals and the like. Radiopaque materials include releasable and non-releasable agents which render the prosthesis visible in any known imaging technique such as X-ray radiographs, magnetic resonance imaging, computer assisted tomography and the like. The radiopaque material may be any conventional radiopaque material known in the art for allowing radiographic visualization of a prosthesis, and may be, e.g., metal wire or flakes made from a biocompatible material, such as titanium, tantalum, stainless steel, or nitinol; or metallic salts (such as barium compounds).

Medicinal agents may be incorporated into the prosthesis at various points in the manufacturing process. For example, a suitable medicinal agent can be mixed with a fluid absorbing liquid polymer before it is cured or fixed. Alternatively, a suitable medicinal agent may be dissolved into a solvent cast solution and then diffused into the hydrogel in accordance with normal kinetic principles. If the prosthesis is then dehydrated, the medicinal agent collects in the interstices of the hydrogel.

A prosthesis according to the disclosure herein may be sterilized by any suitable conventional means, e.g., ethylene oxide, irradiation, etc. and packaged for distribution. A kit containing the sterilized prosthesis and a package insert describing the trapezium prosthesis, along with instructions is useful for medical practitioners.

Techniques for implanting trapezium prostheses in hands are well-known. It is contemplated that the trapezium prosthesis may be implanted in the fully hydrated state to allow visualization of the prosthesis in the void created by removal of the native trapezium. In the present case, minimally invasive implantation techniques are improved and facilitated by the reduced dimension and overall configuration of the reduced configuration. In addition, the ability to provide custom implantation shapes allows an optimal insertion shape to be manufactured. For implantation of the trapezium prosthesis into a hand, access to carpus may, e.g., be performed dorsally. Indeed, palmar, dorsal, medial, and lateral surgical approaches can all be performed. The prosthesis is designed to reduce tissue trauma due to surgery by allowing implantation of the prosthesis in a reduced configuration, thus allowing for minimally invasive surgical techniques. The ability to assume a state of reduced configuration advantageously minimizes undue disruption to soft tissue.

In practice, the bony segments are exposed, care being taken to preserve as much as possible soft tissue connections. The first metatarsal can be surgically sculpted to receive the end of the stem, care being taken again to avoid damage to soft tissue connections. Surgical access may also be gained to the marrow cavities of the bone, and the stem implanted in the marrow cavity, optionally using bone cement or other attachment modalities. It may be appropriate to cause the stem of the prosthesis to fit fairly tightly in the marrow cavity of the bone in which it is implanted. For example, as the prosthesis swells in an intramedullary canal, pressure and/or compression act to frictionally engage the walls of the canal and fix the prosthesis in place. In embodiments, the unconstricted volume of the stems of the trapezium joint, when expanded (also referred to herein as the second configuration), is slightly greater than the space created in the intramedullary canal such that, in situ, the stem of the prosthesis is compressed when situated in the canal. In this manner, the swellable stem exerts positive pressure against the walls of the canal, thus maintaining pressure within the canal and securing the stem in place by virtue of the friction created thereby. In embodiments, the surface of the stem are shaped to encourage frictional affixation and/or treated to encourage bone ongrowth or ingrowth, as by providing, e.g., the stem with barbs, spurs or spikes and/or porous surfaces, by applying to the stem a growth factor, cell adhesion promoter such as collagen or the like, etc.

A fastener, such as a suture may be used to position and secure the prosthesis in place. The reinforcement member may be used as a buttress or pledget to secure the fastener to the prosthesis. As mentioned above, the reinforcement member may be preloaded with the fastener, e.g., a suture, extending out of the implant which is then used to anchor the prosthesis to surrounding bone or tissue. When the reinforcement member is not preloaded, a surgeon may pass a fastener such as a suture through any portion of the outside face of the implant and through the reinforcement member, e.g., a mesh or around a bar or beam, enclosed in the implant. The intended anatomy for the sutures is simply the surrounding tissues and ligaments. If possible, the fastener(s) could be used to repair and close the joint capsule but if not possible simply suturing the implant to the surrounding tissue in such a way as to not limit function is all that is intended.

It should be understood that the examples and embodiments provided herein are preferred embodiments. Various modifications may be made to these examples and embodiments without departing from the scope of the disclosure which is defined by any appended claims. For example, those skilled in the art may envision additional polymers and/or hydrogels which can be compacted and shaped according to the techniques described herein. Similarly, the shapes of the hydrated or expanded trapezium prosthesis described herein are exemplary and any suitable expanded prosthesis shape can be subjected to the techniques described herein to create an optimally shaped, substantially dehydrated prosthesis for minimally invasive insertion into the implantation space. Although especially well-suited for implantation in a dehydrated state, the prosthesis can be delivered in a hydrated state at the time of implantation. Moreover, those skilled in the art can envision additional radially collapsible members for exerting substantially uniform radial compression on the prosthesis which are not set forth herein. In addition, process parameters such as temperature, humidity, pressure, time and concentration may be varied according to conventional techniques by those skilled in the art to optimize results. These are merely examples of many modifications those skilled in the art may make.

What is claimed is:

1. A trapezium prosthesis comprising a swellable fluid absorbing polymer dimensioned and configured to fit into a space vacated by removal of at least a portion of a natural trapezium.

2. The trapezium prosthesis according to claim 1 wherein the swellable fluid absorbing polymer is a hydrogel.

3. The trapezium prosthesis according to claim 2 wherein the prosthesis has a lubricious surface when the hydrogel is at least partially hydrated.

4. The trapezium prosthesis according to claim 1 wherein the prosthesis has a first dehydrated configuration and a second at least partially hydrated configuration, the first configuration being of reduced size relative to the second configuration.

5. The trapezium prosthesis according to claim 1 wherein the prosthesis has a frustocylindrical shape, the frustocylindrical shape including a frustum and an oppositely disposed planar base, the frustum being canted relative to a planar base.

6. The trapezium prosthesis according to claim 5 further comprising a stem projecting outwardly from the frustum, the stem being dimensioned and configured to be inserted into a canal formed in a first metatarsal bone.

7. The trapezium prosthesis according to claim 1 further comprising an internal reinforcement member.

8. The trapezium prosthesis according to claim 8 wherein the reinforcement member is adapted to receive a fastener for affixing the prosthesis to surrounding hard or soft tissue.

9. The trapezium prosthesis according to claim 8 wherein the reinforcement member has a fastener attached thereto, the fastener extending out of the prosthesis, the fastener adapted to fasten the prosthesis to surrounding hard or soft tissue.

10. The trapezium prosthesis according to claim 1 having a modulus of elasticity of from about 1 MPa to about 10 MPa.

11. The trapezium prosthesis according to claim 1 further comprising a medicinal agent.

12. A trapezium prosthesis comprising a body having a frustocylindrical shape, the frustocylindrical shape including a linear frustum and an oppositely disposed planar base, the frustum being canted relative to a planar base.

13. The trapezium prosthesis according to claim 12 further comprising a stem projecting outwardly from the frustum, the stem being dimensioned and configured to be inserted into a canal formed in a first metatarsal bone.

14. The trapezium prosthesis according to claim 12 further comprising an internal reinforcement member.

15. The trapezium prosthesis according to claim 14 wherein the reinforcement member is adapted to receive a fastener for affixing the prosthesis to surrounding hard or soft tissue.

16. The trapezium prosthesis according to claim 14 wherein the reinforcement member has a fastener attached thereto, the fastener extending out of the prosthesis, the fastener adapted to fasten the prosthesis to surrounding hard or soft tissue.

17. The trapezium prosthesis according to claim 14 wherein the prosthesis is made of a swellable fluid absorbing polymer.

18. The trapezium prosthesis according to claim 17 wherein the swellable fluid absorbing polymer is a hydrogel.

19. The trapezium prosthesis according to claim 12 wherein a surface of the prosthesis is lubricious.

20. The trapezium prosthesis according to claim 17 wherein the prosthesis has a first dehydrated configuration and a second at least partially hydrated configuration, the first configuration being of reduced size relative to the second configuration.

21. The trapezium prosthesis according to claim 17 having a modulus of elasticity of from about 1 MPa to about 10 MPa.

22. The trapezium prosthesis according to claim 12 further comprising a medicinal agent.
